(19) United States
(12) Patent Application Publication
(10) Pub. No.: US 2006/0211073 A1
(43) Pub. Date: Sep. 21, 2006

(54) Assay for B-Raf activity based on intrinsic MEK ATPase activity

(76) Inventors: Earl W. May, Upper Providence, PA (US); Cynthia M. Rominger, Upper Providence, PA (US); Michael D. Schaber, Upper Providence, PA (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/084,993

(22) Filed: Mar. 21, 2005

(51) Int. Cl. C12Q 1/48

(52) U.S. Cl. 435/15

(57) ABSTRACT

The present invention provides a convenient assay to identify compounds with B-Raf inhibitory activity, referred to as the BRAMA (B-Raf Accelerated MEK ATPase) assay. The BRAMA assay is based on the discovery of ATPase activity of MEK, and utilizes changes in NADH concentration over time as an indicator of the production of ADP by activated MEK ATPase, where the MEK ATPase activity is activated by B-Raf. NADH concentration may conveniently be measured by Optical Density.

### FIELD OF THE INVENTION

The present invention relates to a catalytic assay of B-Raf activity that is based on intrinsic ATPase activity of MEK kinase. The assay is referred to as the BRAMA assay (B-Raf Accelerated MEK ATPase), and is suitable for use in identifying and characterizing inhibitors of B-Ref.

### SUMMARY OF THE INVENTION

A first aspect of the present invention is a method of screening a test compound to detect B-Raf inhibitory activity, where a reaction mixture is provided containing B-Raf, MEK and ATP in the presence of a test compound, under conditions that would allow phosphorylation of MEK by B-Raf in the absence of any B-Raf inhibitor. NADH concentration in the reaction mixture is measured over time, where increased NADH concentrations compared to the NADH concentrations that would be detected in the absence of any B-Raf inhibitor indicates the test compound has B-Raf inhibitory activity.

### BACKGROUND OF THE INVENTION

One of the primary routes of signal transduction for cellular growth, apoptosis, and differentiation involves the utilization of the Ras-Raf-MEK-ERK phosphorylation cascade (for reviews, see Bollag et al., Curr Opin Investig Drugs 4:1436 (2003), Lee and McCubrey, Expert Opin Ther Targets 6:659 (2002); Wellbrock et al., Nat Rev Mol Cell Biol 5:875 (2004)). Growth factors and mitogens bind to cell surface receptors that activate Ras which, in turn, is responsible for the initiation of Raf activation. The Raf kinases phosphorylate the MEK kinases, which then activate ERK. Phosphorylated ERK proteins can enter the nucleus and subsequently modulate transcription factors.

This MAPK pathway (mitogen activated protein kinase) has been implicated in human cancers. Ras is a common oncogene, and ERK levels are elevated in many cancers. Recent genotypic analyses, however, identified B-Raf in particular as an interesting oncogenic target. (Davies et al., Nature 417(6892):949 (2002)) About 70% of all malignant melanomas and 15% of all colon cancers contain an activating mutation within the B-Raf enzyme; 90% of all of these mutant proteins contain a glutamic acid substitution for the valine at position 600: V600E (formerly numbered as V599E). These findings have stimulated the search for inhibitors of B-Raf, and especially inhibitors of the V600E oncogenic form of the kinase, as a potential treatment for human cancer.

A variety of assays have been developed to probe the activity of the Raf kinases, but very few have proven to be ideal for high throughput characterization of potential inhibitors. Low throughput methods using polyacrylamide gel electrophoresis have been utilized in primary studies of the enzyme. Quantitation of signal through autoradiography (Lange-Carter and Johnson, Methods Enzymol 255:290 (1995); Xu et al., J Biol Chem 276:26509 (2001), Yan et al., J Biol Chem 269(29):19067 (1994)), liquid scintillation counting (Crews and Erikson, Proc Natl Acad Sci USA 89:8205 (1992), Huang et al., Proc Natl Acad Sci USA 90:10947 (1993), Burack and Sturgill, Biochemistry 36:5929 (1997)), and Western analysis (Soga et al., J Biol Chem 273:822 (1998), Bondzi et al., Oncogene 19:5030 (2000)) have yielded important information about activation and substrate specificity. Filter binding assays have also been successful, but are limited by the apparent low activity of the B-Raf enzyme (Alessandrini et al., Proc Natl Acad Sci USA 89:8200 (1992)., Alessi et al., EMBO J. 13:1610 (1994)). High amounts of the enzyme in the reaction are necessary to yield sufficient signal over background, thus high sensitivity is difficult to attain. In addition, there has been little reported success in isolating a peptide substrate of B-Raf with suitable activity (Force et al., Proc Natl Acad Sci USA 91:1270 (1994)). A variety of coupled assay formats have been developed to capitalize on the amplification afforded by the downstream kinases MEK and ERK. These have various end products for quantitation, including filter binding of radiolabeled myelin basic protein (Alessi et al., Methods Enzymol. 255:279 (1995), Stokoe and McCormick, EMBO J. 16:2384 (1997)), ELISA analysis of ERK itself (Mallon et al., Anal Biochem. 294:48 (2001)), and scintillation proximity assays using a peptide substrate for ERK (McDonald et al., Anal Biochem. 268:318 (1999)) or one of ERK's natural substrates, stathmin (Antonsson et al., Anal Biochem. 267:294 (1999)).

However it can be difficult to isolate and characterize B-Raf activities in the context of these multi-protein cascade assays. They can be costly in reagents and necessitate follow-up deconvolution assays to determine the actual targets of small molecule inhibition.

While exploring assay formats that examine the other product of the kinase reaction, i.e. ADP, the present studies revealed that MEK-1 , a substrate for B-Raf, contained a robust intrinsic ATPase activity that was dependent upon activation by B-Raf. Such ATPase activities have been previously identified in other kinases, including protein kinase A (Moll and Kaiser, J Biol Chem. 251:3993 (1976)), protein kinase C (O'Brian and Ward, Biochemistry. 29:4278 (1990); Ward and O'Brian, Biochemistry 31:5905 (1992)), hexokinase (Mulcahy et al., Anal Biochem. 309:279 (2002)), phosphorylase kinase (Paudel and Carlson, J Biol Chem. 266:16524 (1991)), and ERK (Prowse and Lew, J Biol Chem. 276:99 (2001)). While the biological relevance of the intrinsic MEK-1 ATPase was not elucidated, this activity nevertheless can be used in assaying B-Raf catalysis, as described herein.

### BRIEF DESCRIPTION OF THE DRAWINGS

FIG. 1 provides a schematic of the pathway utilized in the BRAMA assay system, where B-Raf catalyzes the reaction between MEK and ATP, producing phospho-MEK (activated MEK) which possesses an intrinsic ATPase activity and resulting in the production of ADP and inorganic phosphate (Pᵢ). Pyruvate kinase combines ADP and PEP to make pyruvate. Lactate dehydrogenase makes lactate from pyruvate, oxidizing NADH in the process. The drop in NADH concentration is thus a direct indication of the production of ADP by activated MEK.

FIG. 2. A series of experiments were conducted using the PK/LDH system to monitor the production of ADP in the reaction by monitoring the drop in NADH absorbance at 340 nm. Squares: negative control (neither MEK nor B-Raf added to the reaction). Circles: 80 nM V600E B-Raf. Triangles: 1000 nM MEK. Diamonds: 80 nM V600E B-Raf and 1000 nM MEK. Note: circles representing 80 nM V600E B-Raf are difficult to distinguish on FIG. 2 as they overlap with the squares and triangles.

FIG. 3: Titration of V600E into BRAMA. Various concentrations of V600E B-Raf were delivered to BRAMA reactions with 300 nM MEK-1. B-Raf concentrations from 0 to 4000 pM are indicated in FIGS. 3A-3D. 3A: Progress curves from the BRAMA reaction with PK/LDH coupling. 3B: First derivative plots of progress curves from B-Raf titrations. Slopes were determined from the 13 nearest neighbors in the original progress curves (GraphPad Prism “Differentiate” function). 3C: Acceleration coefficients from the fit of the progress curves to a second order polynomial are plotted as a function of B-Raf concentration. Inset: focus on acceleration from 0 to 0.5 nM B-Raf. 3D: Progress curves from the BRAMA reactions coupled to the PNP/MESG system for inorganic phosphate detection.

FIG. 4. Titration of MEK-1 into BRAMA. Varying concentrations of MEK-1 enzyme were added to the BRAMA reaction. 4A: Four different concentrations of MEK-1 (88-700 nM) were delivered to 10 nM B-Raf in the PK/LDH coupled system. 4B: First derivative plots of progress curves from the MEK titrations shown in FIG. 4A. Slopes were determined from the 13 nearest neighbors in the original progress curves (GraphPad Prism “Differentiate” function). 4C: Various concentrations of activated phospho-MEK-1 protein (1.8 to 5.6 nM) were delivered to the PK/LDH-coupled system in the absence of any B-Raf. 4D: Phospho-MEK-1 reactions without B-Raf (as in 4C) were analyzed using the PNP/MESG system for inorganic phosphate detection.

FIG. 5. The behavior of inhibitors specific to B-Raf and MEK in the BRAMA reaction. Inhibitors specific to B-Raf (SB-590885) and MEK (U0126) were delivered to the PK/LDH-coupled BRAMA reaction. 5A: Progress curves from a titration of the B-Raf inhibitor SB-590885 into a reaction of 0.25 nM B-Raf and 300 nM MEK. Concentrations of SB-590885 are indicated in nM (0-50 nM). 5B: Concentration-response curves from two independent experiments as described in FIG. 5A. Data were fit as described in Materials and Methods to yield an IC50 of 960 pM (±69) and a Hill slope of 0.98 (±0.06). 5C: Progress curves of the MEK-specific inhibitor U-0126 (0-12,500 nM) delivered to a reaction of 80 nM B-Raf and 300 nM MEK.

FIG. 6. Correlations of BRAMA to other B-Raf activity assays. 6A: Western blot of a titration of a compound with known potent B-Raf inhibitory activity into a BRAMA assay. In the left panel, an anti-MEK primary antibody was used with from 10,000 nM to 0.03 nM of the B-Raf inhibitor. In the right panel, an anti-phosphoMEK primary antibody was used, also with from 10,000 nm to 0.03 nM of the B-Raf inhibitor. 6B: IC₅₀s derived from quantitation of Western blot analyses of five different compounds with known B-Raf inhibitory activity are compared to their IC₅₀s (nM) derived from BRAMA assay. 6C: Correlation of IC₅₀s (nM) generated from BRAMA and from filter-binding experiments (as described in Example 1 herein), for multiple compounds known to have B-Raf inhibitory activity.

### DETAILED DESCRIPTION

The present studies have revealed that MEK-1 kinase possesses an intrinsic ATPase activity. As is true with its kinase function, the MEK-1 ATPase activity is entirely dependent upon the phosphorylation state of the enzyme: unphosphorylated MEK-1 does not consume ATP, while phosphorylated MEK-1 can hydrolyze ATP. MEK kinase activities are modulated in vivo by Raf kinases, and thus activation of MEK-1 by, for example, B-Raf can be monitored through the increase in the intrinsic ATPase activity of MEK-1 in vitro. Data are presented herein to show the velocity of the MEK-1 ATPase is dependent upon the concentration of MEK-1 in the reaction, but the acceleration of the ATPase signal is directly proportional to B-Raf-dependent phosphorylation of MEK-1. Based upon these findings a sensitive and robust assay for B-Raf activity, based on this intrinsic ATPase activity in the MEK-1 kinase, was developed. The assay is referred to as the BRAMA assay (B-Raf Accelerated MEK ATPase), and is suitable for use in identifying and characterizing inhibitors of B-Ref.

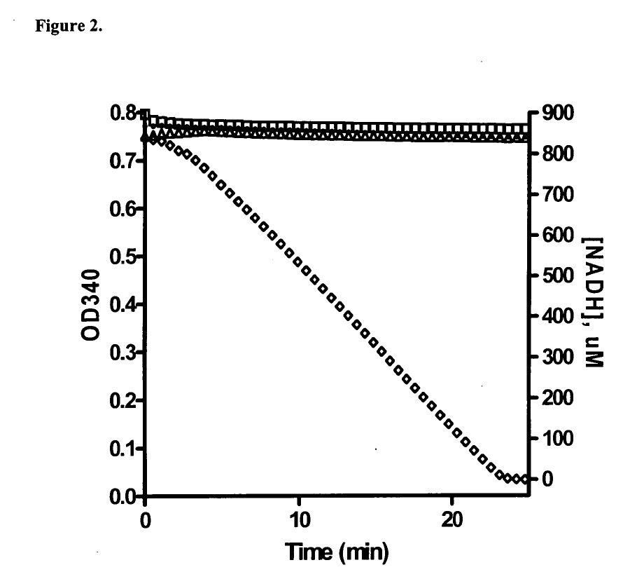

Due to the low sensitivity and throughput of existing B-Raf catalytic screens, the present researchers began to pursue alternative assay formats. One such format involved following the production of ADP in the B-Raf/MEK reaction rather than the more typically monitored phosphorylated protein product. The addition of pyruvate kinase (PK) and lactate dehydrogenase (LDH) to the reaction converts the coupling substrate phosphoenol pyruvate (PEP) to lactate with the concomitant oxidation of NADH, a cofactor which can be followed spectrophotometrically (Webb M R, Proc Natl Acad Sci USA 89:4884 (1992)). Experiments utilizing a kinase-dead derivative of MEK-1 (K97R) as a substrate were not successful in yielding signal in this assay (data not shown). However, a control experiment that utilized wild-type, unphosphorylated (inactive) MEK-1 as a B-Raf substrate yielded a surprising result. As can be seen in FIG. 2, when both MEK-1 and B-Raf were present there was a dramatic drop in absorbance at 340 nm over time, suggesting the presence of a potent ADP-generating activity. Each mole of NADH consumed in the reaction corresponded to one mole of ADP produced. Thus the consumption of 900 μM ADP in less than thirty minutes could not be explained by the phosphorylation of 1 μM MEK by B-Raf, and the ADP must have been generated in some other fashion. It was hypothesized that there existed an intrinsic ATPase activity within phospho-MEK-1 in the absence of downstream protein substrates of MEK-1 (e.g. ERK).

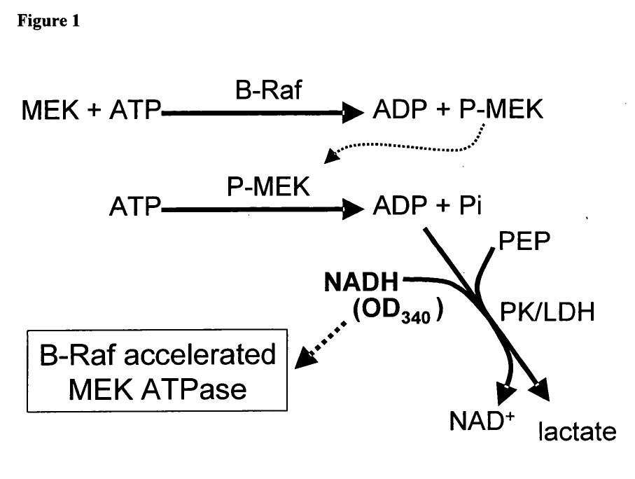

As shown in FIG. 1, it has been determined that B-Raf catalyzes the reaction between MEK and ATP, producing phospho-MEK and ADP. This activation of MEK turns on an intrinsic ATPase activity, producing ADP and inorganic phosphate (Pᵢ). Pyruvate kinase combines ADP and PEP to make pyruvate. Lactate dehydrogenase makes lactate from pyruvate, oxidizing NADH in the process. The drop in NADH concentration, as monitored at 340 nm, is thus a direct indication of the production of ADP by activated MEK.

### Description of the Present Assay

The present research provides a convenient assay to identify compounds having B-Raf inhibitory activity; the assay is referred to as the BRAMA (B-Raf Accelerated MEK ATPase) assay. The BRAMA assay is particularly useful in screening one or more compounds to identify any that have B-Raf inhibitory activity.

The BRAMA assay as described herein is based on the discovery of ATPase activity of MEK, and utilizes changes in NADH concentration over time as an indicator of the production of ADP by activated MEK ATPase, where the MEK ATPase activity is activated by B-Raf. MEK is also referred to as MAPKK (for MAPK (mitogen-activated protein kinas) Kinase) or MKK. Two forms of MEK are known, MEK-1 and MEK-2. Both are kinased and activated by Raf proteins.

In the BRAMA assay, NADH concentration can be measured by any suitable means as known to one of skill in the art. Measurement of Optical Density at 340 nm is one method of detecting NADH concentration. Alternatively the production of inorganic phosphate may be measured and used as an indicator of B-Raf inhibitory activity, for example as described herein using the PNP/MESG system in which 2-amino-6-mercapto-7-methyl-purine riboside (MESG) is used as a substrate instead of NADH, and purine nucleoside phosphorylase enzyme (PNP) is used as the coupling enzyme instead of PK/LDH, and inorganic phosphate is monitored over time by following the increase of the absorbance at 360 nm of the MESG cleavage product 2-amino-6-mercapto-7-methyl-purine.

The BRAMA assay to screen compounds for B-Raf inhibitor activity can be carried out by providing a reaction mixture containing B-Raf, MEK, ATP, and a test compound, under conditions that allow phosphorylation of MEK by B-Raf. NADH concentration over time is monitored and the presence of a compound with B-Raf inhibitor activity is determined by comparing changes in NADH concentration to the changes in NADH concentration that would be expected in the absence of any B-Raf inhibitor. The presence of B-Raf inhibitory activity is indicated when the decrease in NADH concentration over time is less than (reduced compared to) the decrease in NADH which would occur in the absence of B-Raf inhibition. In other words, the presence of a B-Raf inhibitor in the BRAMA assay will result in increased NADH concentration compared to a control assay (no B-Raf inhibitor), when compared under similar test conditions and at a similar time point in the assay.

NADH concentration changes in a BRAMA assay using a test compound or known B-Raf inhibitor can be compared to a control assay run at approximately the same time; to the results of a control assay that had been run previously or was run at a later date; or to a standard that was previously determined and provided in written form, e.g., as a chart, graph, or numerical table.

As described further herein, B-Raf inhibitory activity is indicated where a test compound affects the acceleration of the progress curve(s). Where the terminal velocity of the progress curve(s) are affected by a test compound, p-MEK (activated MEK) inhibitory activity is indicated. In the event it is questioned whether the results of the BRAMA assay indicate p-MEK or B-Raf inhibition, a follow up assay utilizing p-MEK in the absence of B-Raf can be utilized.

It will be apparent to one skilled in the art that the BRAMA assay can be run with more than one test compound at a time. Where results indicate the presence of a B-Raf inhibitor, the assay can be re-run using smaller subgroups of test compounds or individual test compounds to identify which test compound(s) possess B-Raf inhibitory activity.

The time needed to run a BRAMA assay will vary depending on the concentration of B-Raf and MEK, and other variables as will be apparent to those skilled in the art. In a preferred embodiment, the assay does not take longer than 5 hours to complete. In a more preferred embodiment, the BRAMA assay will be completed in under 4 hours, 3 hours, or 2 hours. It will be apparent to one skilled in the art that the time point(s) at which NADH concentration is measured must encompass a time frame sufficient for the reaction to progress. NADH concentration is preferably measured at multiple points over the course of the assay; alternatively, NADH concentration may be measured at only a few, or a single, suitable time point(s).

In a preferred embodiment, BRAMA assays are run with a B-Raf concentration of between 0.10 and 0.50 nm, preferably about 0.25 nM. However, it will be apparent to those skilled in the art that B-Raf concentration can be reduced further to accommodate the measurement of more potent inhibitors. In a preferred embodiment, BRAMA assays are run using human B-Raf (including human B-Raf containing the V600E polymorphism) and human MEK-1.

Typically, compounds that are small chemical molecules are screened, such as small organic molecules having a molecular weight of from 50 to 2500 daltons. Alternative molecules that may be screened include biomolecules such as steroids, purines, pyrimidines, derivatives, structural analogs or combinations thereof. Such agents are obtained from a wide variety of sources including libraries of synthetic and natural compounds. Further, known pharmacological agents can be subjected to directed or random chemical modifications, such as acylation, alkylation, esterification, amidification, etc. to produce structural analogs that are screened.

### EXAMPLE 1

### Materials and Methods

Chemicals and reagents: ATP was acquired from Amersham Pharmacia Biotech, Inc., #27-2056-01; stock concentration 100 mM pH 7.5, purity >98% in purified H₂O, stored at −b 20° C. 3-N-morpholinopropanesulfonic acid sodium salt (MOPS) was acquired from Sigma-Aldrich, #M-9024; stock concentration 80 mM pH 7.2, stored at 4° C. Tween20 was acquired from Calbiochem, #655204; stock concentration 10%, stored at room temperature. 1,4-dithiothreitol (DTT) was acquired from Roche, #100 034; stock concentration 1 M in H₂O, stored at −20° C. EGTA was acquired from Sigma Aldrich, #E-4378; stock concentration 500 mM in H₂O, pH 7.8, stored at room temperature. Albumin, bovine serum (BSA) was acquired from Sigma Aldrich, #B4287-256; stock concentration 10 mg/ml in H₂O, stored at −20° C. Magnesium chloride, anhydrous (MgCl₂) was acquired from Sigma-Aldrich, #M8266; stock concentration 1 M in H₂O, stored at room temperature. 3-nicotinamide adenine dinucleotide reduced disodium salt hydrate (NADH) was acquired from Sigma-Aldrich, #N6005; stock concentration 30 mM in H₂O, stored at −20° C. Phosphoenol pyruvic acid (PEP) was acquired from Sigma-Aldrich, #P3637; stock concentration 100 mM in H₂O, stored at −20° C. Dimethyl sulfoxide (DMSO) was acquired from Sigma Aldrich, #D8418 and stored at room temperature. Gamma ³³P-ATP (3000 Ci/mmol; 10 mCi/ml) was acquired from Perkin Elmer Life Sciences, #NEG302H001MC and stored at −20° C. Phosphatase Inhibitor cocktail (100×) was acquired from Sigma Chemical Co., #P-2850 and stored at −20° C. β-Glycerol PO₄ was acquired from Sigma Chemical Co., #G-6251 and stored at 4° C. MES was acquired from Sigma Chemical Co., #M-8250. Microscint20 was acquired from Packard BioScience, #6013621.

B-Raf: Full-length His-tagged Human B-Raf V600E clone number DU630, molecular weight 88,479. Acquired from the University of Dundee Kinase Consortium. Purified on 17 Nov. 2003. 0.84 mg/ml, purity 80% (B-Raf concentration=7590 nM). Stored at −80° C. in B-Raf storage buffer: 50 mM Tris-HCl pH 7.5, 270 mM sucrose, 150 mM NaCl, 0.1 mM EGTA, 0.1% BME, 0.02% Brij 35, 1 mM benzamidine, 0.2 mM PMSF.

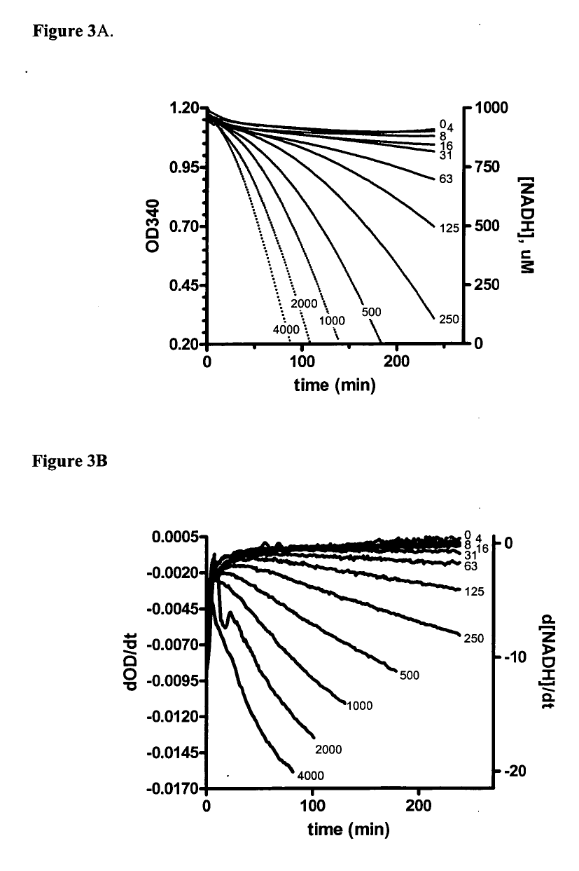

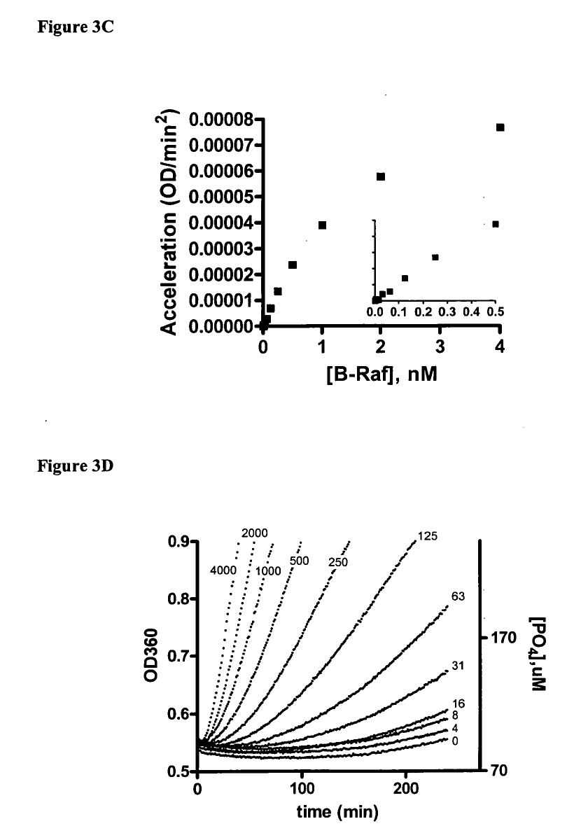

Unactivated MEK-1: Two sources of unactivated MEK-1 were used. The first (used for FIGS. 2, 4A, and 5C) was a human derivative from Upstate (Waltham, Mass.) (#14-420, lot #25557AU) with an N-terminal GST tag and a C-terminal His₆ tag, molecular weight 71 kDa. It was stored at −80° C. in 14 μM in 50 mM Tris-HCl pH 7.5, 0.1 mM EGTA, 0.03% Bris-35, 5% glycerol, 0.1% 2-mercaptoethanol, 0.2 mM PMSF, 1 mM benzamidine. The second (used in FIGS. 3, 5A, and 6) was human MEK-1 from plasmid 39179 (BioCat GmbH, Heidelburg, Germany), molecular weight 43,415. It was over-expressed in E. coli BL21 [DE3]/pRR692 as a fusion to GST (N-terminal). After purification on glutathione sepharose-4FF, the GST tag cleaved was released with TEV protease and then the MEK-1 further purified by another run on glutathione sepharose and sizing on Superdex-200. 2 mg/ml, purity˜76% (MEK-1 concentration=35 μM). Stored at −80° C. in PBS+5% glycerol.

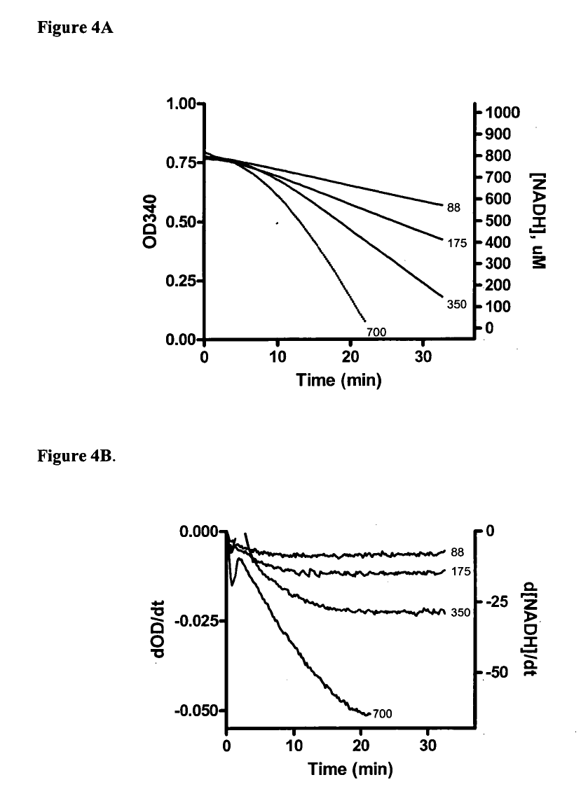

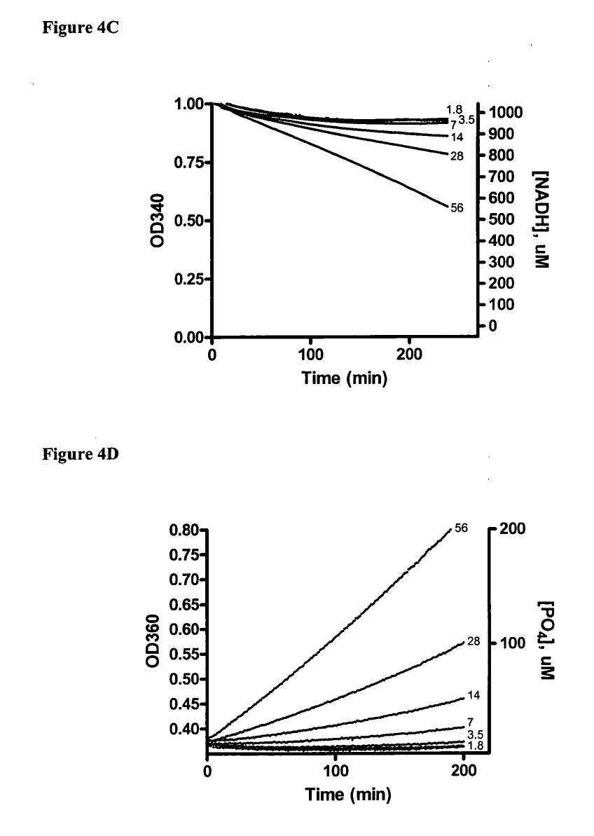

Activated MEK-1 : Phospho-MEK (used in FIGS. 4C and 4D) was acquired from Upstate (Waltham, Mass.) (#14-429, lot #26232U) with an N-terminal GST tag and a C-terminal His₆ tag, molecular weight 71 kDa. It was activated using c-Raf and then re-purified using nickel/NTA agarose, stored at −80° C. in 14 μM in 50 mM Tris-HCl pH 7.5, 0.1 mM EGTA, 0.03% Bris-35, 5% glycerol, 0.1% 2-mercaptoethanol, 0.2 mM PMSF, 1 mM benzamidine.

kdMEK: GST-tagged human kinase-dead MEK-1 (K97R), molecular weight 70,403. Over-expressed in E. coli and purified on glutathione sepharose-4FF. 15.7 mg/ml, purity˜50% (GST-kdMEK concentration=112 μM). Stored at −80° C. in 50 mM Tris-HCl pH 8.0, 100 mM NaCl, 1 mM EDTA, 1 mM Benzamidine, 10 mM BME, 0.02% Brij 35, 5% glycerol.

PK/LDH: from rabbit muscle acquired from Sigma Aldrich Catalog #P0294—5 ml as a 100× stock (1000 U/ml PK, 700 U/ml LDH). Stored at −20° C. in buffered aqueous glycerol solution (50%).

Instruments and Equipment: A Molecular Devices Spectramax Plus was used to monitor NADH absorbance at 340 nm, or MESG absorbance at 360 nm. Kinetic reads were conducted up to four hours with timepoints every eleven to thirty seconds.

For filter binding experiments a Millipore vacuum apparatus was used, followed by a Top Count NXT HTS, Perkin-Elmer.

Some compound dilutions were performed on a WellPro Pro-max dilutor.

For BRAMA experiments, U-bottom, clear polystyrene 96-well reaction plates were acquired from USA Scientific Plastics, Greiner #5665-0101. For filter binding experiments several plates and adaptors were used: polypropylene plates 96 well round bottom, Costar catalog #3359; Non-Binding Surface plates, Costar catalog #3884; MAPH plates with phosphocellulose filter, Millipore catalog #MAPHN0B50; and Multi screen Top Count Adapters, Millipore catalog #SE3M203V6.

BRAMA Method:

If required, B-Raf was diluted in B-Raf dilution buffer (20 mM MOPS pH 7.2, 0.01% Tween20, 1 mM DTT, 5 mM EGTA, 0.1 mg/ml BSA, 10 mM MgCl₂).

The reaction was assembled in two parts. First, an appropriate amount of 2× Enzyme Mix is prepared: 0.5 nM V600E B-Raf (or as indicated), 20 mM MOPS pH 7.2, 0.01% Tween20, 1 mM DTT, 5 mM EGTA, 0.1 mg/ml BSA, 10 mM MgCl₂. Then the appropriate amount of 2× Substrate Mix was prepared: 0.6 μM MEK (or as indicated), 2 mM ATP, 20 mM MOPS pH 7.2, 0.01% Tween20, 1 mM DTT, 5 mM EGTA, 0.1 mg/ml BSA, 10 mM MgCl₂, 1.8 mM NADH, 2 mM PEP, 2× PK/LDH.

2.5 μl of compound dilutions in 100% DMSO were delivered to the bottom of each well of a dry reaction plate. Following the addition of 22.5 μl of 2× Enzyme Mix to each well of reaction plate, the reaction was started by delivery of 25 μl of 2× Substrate mix to each well of reaction plate. Reaction progress was monitored on a Spectramax Plus plate reader at room temperature for four hours. Optical Density (OD) is read OD at 340 nm every 30 seconds, or as reported.

Final reaction conditions: 0.25 nM V600E B-Raf (or as reported), 300 nM MEK (or as reported), 1 mM ATP, 20 mM MOPS pH 7.2, 0.01% Tween20, 1 mM DTT, 5 mM EGTA, 0.1 mg/ml BSA, 10 mM MgCl₂, 900 μM NADH, 1 mM PEP, 10 U/ml PK, 7 U/ml LDH, 5% DMSO.

Data analysis: Progress curve data for each plate were obtained. Points in first twenty minutes were excluded from analysis. Timepoints beyond depletion of NADH were also excluded. A plot of the slopes as a function of time [dOD/dt] was inspected to (a) verify the linearity of the B-Raf reaction over the timecourse, and (b) verify that the terminal MEK reaction rate had not been reached—as would be indicated by a plateau in the curve. Progress curves [OD=f(t)] were fit to a second-order polynomial: y=A+Bx+Cx². The acceleration coefficients (C) were tabulated, and the percent maximal acceleration due to B-Raf was plotted as a function of inhibitor concentration to yield a concentration-response curve. Three parameter fits are conducted on the concentration response curves (min fixed to zero; max, IC₅₀, and slope all float). IC₅₀s and Hill Slopes were recorded with their associated standard error. The results of the average of replicate experiments with test compounds are reported, along with their standard deviations.

Phosphate Detection

Experiments that were monitored through the detection of inorganic phosphate were conducted with the EnzChek Phosphate Assay Kit from Molecular Probes (Eugene, Oreg.) (E-6646). 1 mM 2-amino-6-mercapto-7-methyl-purine riboside (MESG) and 100 U/ml purine nucleoside phosphorylase enzyme (PNP) were prepared per instructions and frozen away as aliquots at −20° C. Reactions were prepared as for the standard BRAMA assay with the following changes. 400 μM MESG was used as a substrate instead of 900 μM NADH, and 1 U/ml PNP was used as the coupling enzyme instead of 1× PK/LDH. Inorganic phosphate evolution was monitored over time by following the increase of the absorbance at 360 nm of the MESG cleavage product 2-amino-6-mercapto-7-methyl-purine.

Filter Binding Assays:

Final assay conditions: 40 nM V600E B-Raf, 3 μM kinase-dead (kd) MEK-1 , 2.5 μCi/well gamma ³³P-ATP, 10 mM MgCl₂, 1 mM DTT, 1% Phosphatase Inhibitor cocktail, 20 mM MES buffer pH 6.5, 25 mM β-Glycerol PO₄, 5 mM EGTA, 5% DMSO.

MAPH plates were wetted with 200 μl of 0.5% H₃PO₄ for a minimum of 30 minutes. Compound dilutions were conducted in 100% DMSO at 20× final concentration in assay in polypropylene plates. Compounds tested at 11 concentrations, ½ log serial dilution starting at a final concentration of 10 μM. 1.04× assay buffer contained MES, B-Glycerol PO4, EGTA and phosphatase inhibitor cocktail. Mg, ATP, kdMEK and B-Raf were each prepared at 4.16× conc. in 1.04× assay buffer. 2.5 μl compound dilutions were added to Non-Binding Surface (NBS) plate followed by 12 μl/well reagents in the following order: Mg/ATP/B-Raf/kdMEK (addition of kdMEK starts reaction).

Reactions were incubated at room temperature for 20 minutes and stopped in the NBS plate with equal volumes of 1% H₃PO₄ (final H₃PO₄ concentration 0.5%). The stopped reaction was incubated at room temperature for 10 to 20 min and then 85 μl (85%) was transferred to pre-wetted filtered MAPH plates. Following 30 minutes at room temperature to allow for binding of phosphorylated MEK to the filter, the stopped reactions were filtered and the plate washed four times with 0.5% H₃PO₄. Excess wash solution was removed by blotting on a paper towel, and then the filters were dried at 50° C. for 20 to 30 min. After an adapter was added to the reaction plate 50 μl of scintillation cocktail (MicroScint 20) was added and the plates shaken for 1 min. Counts were read in a TOP-Count (count time, 1 min/well, cpm normalized for ³³P).

Data analysis: cpm per well were analyzed using PRISM Graph-Pad. For concentration-response assays, the results of each test well were expressed as y=%Activity. Normalization Equations: y=100*((U1-C2)/(C1-C2)) where U1 is the signal observed in a reaction sample well, C1 is the signal observed in the absence of any added inhibitor (positive control) and C2 is the signal observed in the reaction quenched with 0.5M EDTA prior to the addition of enzyme (negative control). The values of C1 and C2 were averaged from 4 sample wells each in column 12 of plate. Inhibition data were fit to a 2-parameter equation where the lower data limit is 0 and the upper data limit is 100: y=100/(1+(x/IC50) [symbol] s) where s is the slope factor and x is the concentration of test sample. The equation assumes that y falls with increasing x. The results of the best fit are recorded as pIC50 values. pIC50=−log₁₀(IC50).

Quantitative western blot analysis: 30 μl aliquots from the completed ATPase reactions were added to 10 μl 4× Laemmli sample buffer and incubated for 2 minutes at 80° C. Samples were loaded on 12% Bis-Tris polyacrylamide gels (Invitrogen) and electroblotted to nitrocellulose membranes. Blots were incubated for 30 minutes in blocking buffer (Rockland, Gilbertsville, Pa.) followed by incubation for 16 hours at 4° C. with primary antibodies diluted 1:1000 in TBS-T (50 mM Tris 7.5; 150 mM NaCl; 0.1% Tween 20)+5% BSA. The primary antibodies used were: for pMEK detection, rabbit-anti-pMEK and for total MEK detection, rabbit-anti-MEK (Cell Signaling Technology, Beverly, Mass.). Following the primary antibody incubations the blots were washed in TBS-T and reacted in the dark for 1 hour at 24 C with a 1:5000 dilution of goat-anti-rabbit-AlexaFluor680 (Molecular Probes, Eugene, Oreg.) prepared in 0.5× Rockland blocking buffer. Detection of antibody binding was with the Odyssey Infrared Imaging System (Li-Cor Biosciences, Lincoln, Nebr.), and the intensity of bands was quantified with Odyssey Application Software v1.2. IC50 values were generated by plotting band intensity vs. inhibitor concentration.

### EXAMPLE 2

### Production of ADP in B-Raf/MEK Reaction

Due to the low sensitivity and throughput of existing B-Raf catalytic screens, the present researchers began to pursue alternative B-Raf assay formats. One such format followed the production of ADP in the B-Raf/MEK reaction rather than the more typically monitored phosphorylated protein product. The addition of pyruvate kinase (PK) and lactate dehydrogenase (LDH) to the reaction converts the coupling substrate phosphoenol pyruvate (PEP) to lactate with the concomitant oxidation of NADH, a cofactor which can be followed spectrophotometrically (Webb MR, Proc Natl Acad Sci USA 89:4884 (1992)).

A series of experiments were conducted using the PK/LDH system to monitor the production of ADP in the reaction by monitoring the drop in NADH absorbance at 340 nm. Experiments utilizing a kinase-dead derivative of MEK-1 (K97R) as a substrate were not successful in yielding signal in this assay (data not shown). However, a control experiment that utilized wild-type, unphosphorylated (inactive) MEK-1 as a B-Raf substrate yielded a surprising result. As shown in FIG. 2, the addition of either MEK-1 (triangles) or B-Raf (circles) to the PK/LDH coupled reaction yielded no significant production of ADP over background (no MEK-1 or B-Raf, squares). When, however, both MEK-1 and B-Raf were added (diamonds) there was a dramatic drop in absorbance at 340 nm over time, suggesting the presence of a potent ADP-generating activity. Each mole of NADH consumed in the reaction corresponded to one mole of ADP produced. Thus the consumption of 900 μM ADP in less than thirty minutes could not be explained by the phosphorylation of 1 μM MEK by B-Raf. The ADP must have been generated in some other fashion, and it was hypothesized that there existed an intrinsic ATPase activity within phospho-MEK-1 in the absence of downstream protein substrates of MEK-1 (e.g. ERK).

### EXAMPLE 3

### Titration of B-Raf Affects the Acceleration of ADP Production

To explore the novel ATPase activity of MEK-1 kinase, experiments were conducted at a variety of B-Raf concentrations. Concentrations of V600E B-Raf (from 0 to 4000 pM) were delivered to BRAMA reactions with 300 nM MEK; results are shown in FIGS. 3A-3D

The progress curves of such a titration are displayed in FIG. 3A. With increasing concentrations of B-Raf, the rate of ADP production accelerated, as would be expected in a cascade assay where the product of the reaction of interest is the enzyme responsible for the monitored signal. The linearity of acceleration is more evident in a graph of the slopes of the progress curves as a function of time (FIG. 3B). This plot represents the actual progress of the B-Raf reactions, and there is a linear relationship between the slopes of these initial rates and the concentration of B-Raf in the reaction (FIG. 3C). A similar set of B-Raf accelerated progress curves are observed when inorganic phosphate is monitored rather than ADP (FIG. 3D). Production of inorganic phosphate would not previously have been expected from either the B-Raf or MEK kinase activities; inorganic phosphate is not typically released from such transphosphorylation reactions.

### EXAMPLE 4

### Titration of MEK-1 Affects the Terminal Rate of ADP Production

While varying the concentration of B-Raf affects the acceleration, one would expect that varying the concentration of MEK would change the terminal velocity of the reaction. Protein substrate depletion in the B-Raf reaction results in a constant maximal level of activated MEK, and thus a constant terminal ATPase reaction rate.

As shown in FIG. 4, varying concentrations of MEK enzyme were titrated into the BRAMA reaction. High concentrations of B-Raf (10 nM) were supplied to the reaction to quickly arrive at a terminal velocity for four concentrations of MEK (FIG. 4A; four different concentrations of MEK (88-700 nM) delivered to 10 nM B-Raf in the PK/LDH coupled system). The slopes of these ATPase progress curves as a finction of time (FIG. 4B; slopes determined from the 13 nearest neighbors in the original progress curves (GraphPad Prism “Differentiate” function)) again described the progress curves of the B-Raf catalyzed reaction itself. Protein substrate depletion is indicated by the plateau of the ATPase reaction rates. These terminal ATPase reaction rates are linearly proportional to the input MEK substrate.

Commercially acquired phospho-MEK (activated by c-Raf and then re-purified) also displayed this intrinsic ATPase activity. Reactions containing this pre-activated MEK in the absence of B-Raf are shown in FIG. 4C (various concentrations of activated phospho-MEK protein (1.8 to 5.6 nM) delivered to the PK/LDH-coupled system in the absence of B-Raf). The amount of enzyme that could be delivered to the reaction was limited here by the concentration of phosphoMEK available commercially. Nevertheless, linear progress curves were observed, especially at the highest concentrations of phospho-MEK, and with little lag period at the start of the reaction. Phospho-MEK reactions without B-Raf that were monitored by inorganic phosphate evolution (using the PNP/MESG system) showed a similar set of progress curves, (FIG. 4D) confirming the presence of this ATPase activity in c-Raf activated MEK.

### EXAMPLE 5

### Inhibitors of B-Raf and MEK-1 can be Identified with BRAMA

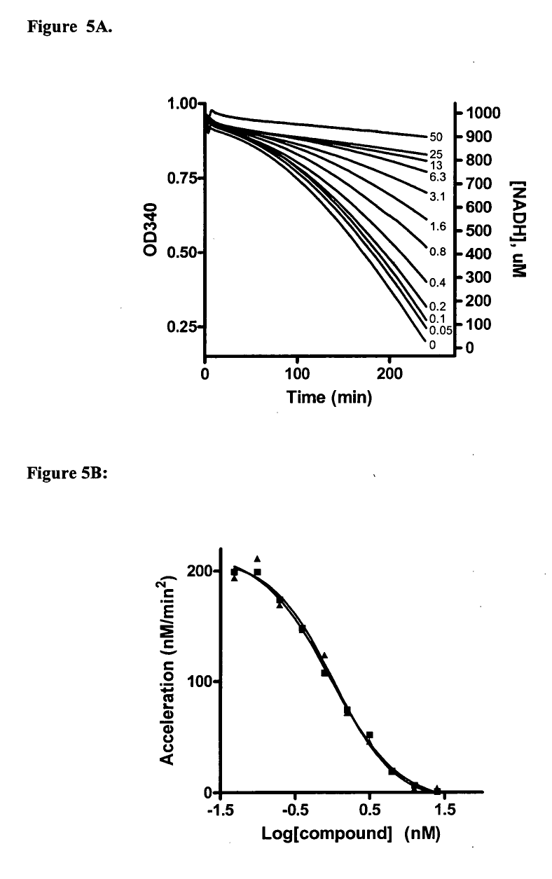

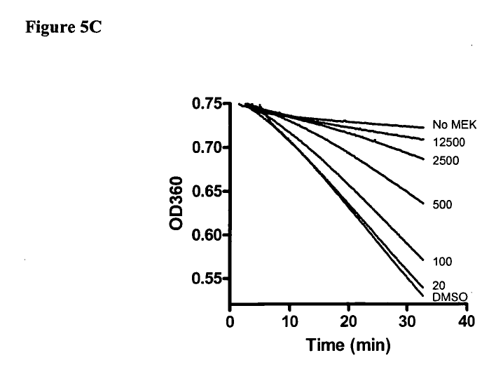

If B-Raf titration results in a change in the acceleration of the MEK ATPase activity, then inhibition of B-Raf should do the same. A potent B-Raf inhibitor, SB-590885 was used in these experiments (see King et al., manuscript in progress: B-Raf(V600E) expression status predicts the anti-tumor response to SB-590885, a potent and selective B-Raf inhibitor). SB-590885 was titrated into the PK/LDH-coupled BRAMA reaction at concentrations ranging from 0.05 nM to 50 nM (FIG. 5A; progress curves from titration of SB-590885 into a reaction of 0.25 nM B-Raf and 300 nM MEK). The acceleration of the MEK ATPase was dramatically affected in a concentration-dependent manner. A concentration-response plot based on the acceleration extracted from each individual progress curve revealed a sigmoidal response with an IC₅₀ of 960 pM (±69), and a Hill slope of 0.98 (±0.06). (FIG. 5B; concentration-response curves from two independent experiments as described for FIG. 5A). This IC₅₀ value compares well to previously established values (0.6 nM, King et al., supra). Separate experiments showed that SB-590885 did not inhibit the ATPase activity of commercially acquired phosphoMEK (IC₅₀>10 μM, data not shown).

Preliminary experiments with the MEK-specific inhibitor U-0126 (Favata et al., J Biol Chem. 273:18623 (1998)) in reactions containing high levels of B-Raf (80 nM) are shown in FIG. 5C (progress curves of U-0126 (0-12,500 nM) delivered to a reaction of 80 nM B-Raf and 300 nM MEK).

This abbreviated concentration-response analysis indicated that the inhibitor affects the terminal velocity of the MEK ATPase reaction. It is expected that the MEK-specific inhibitor will only affect the terminal velocity and have little effect on the B-Raf dependent acceleration of the reaction. Interestingly, the IC₅₀ suggested from this set of inhibitor concentrations (˜500 nM) is significantly higher than published results on the kinase activity of MEK-1. Similar results were obtained with commercially acquired phosphoMEK (data not shown).

### EXAMPLE 6

### BRAMA Correlates with Other B-Raf Catalytic Assays

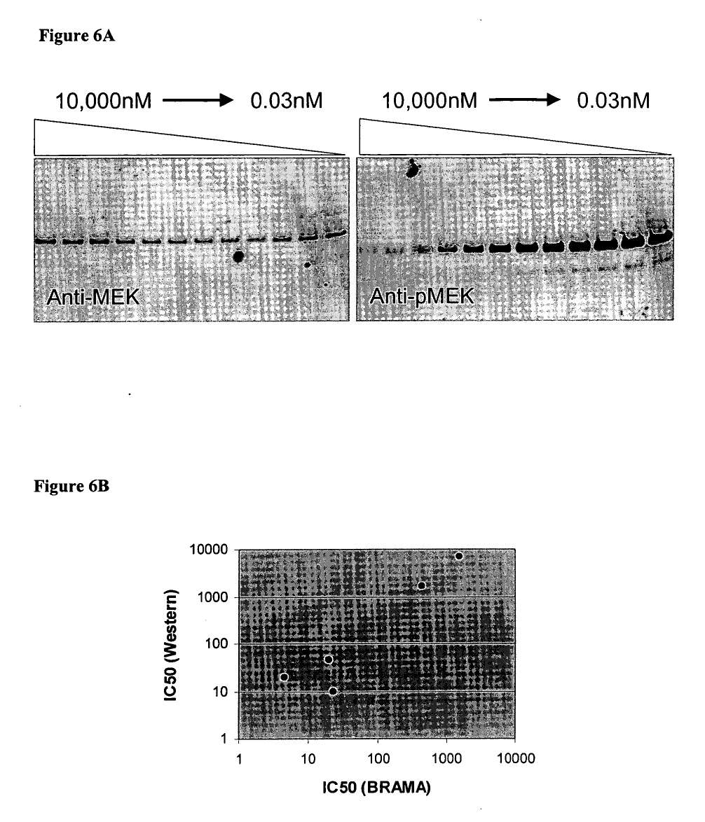

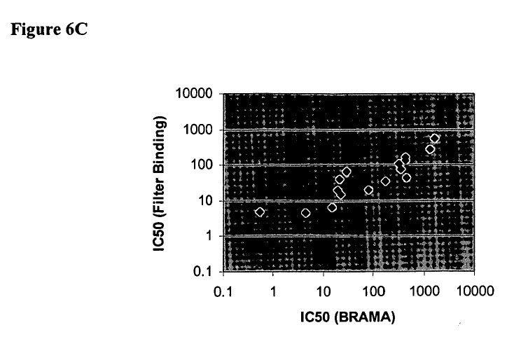

Compound potencies derived from BRAMA assays were compared to the results of two other B-Raf catalytic assays. First, Western analysis was employed to directly visualize and quantify the amount of phospho-MEK generated in a set of reactions containing increasing concentrations of a compound known to have potent B-Raf inhibitory activity (FIG. 6A; Western blot of titration of (10,000 nM to 0.03 nM)). In the left panel of FIG. 6A, the reactions were probed with a MEK-specific primary antibody that recognizes MEK independent of its phosphorylation state. In the right panel of FIG. 6A, the primary antibody is specific to the phosphorylated form of MEK. Normalizing the signal of the phospho-specific antibody to the antibody signal for total MEK, IC₅₀s could be derived (data not shown). The IC₅₀s of a small set of compounds with known B-Raf inhibitory activity were generated and compared favorably to the BRAMA results (FIG. 6B; B-Raf inhibitor IC₅₀s derived from quantitation of Western blot analyses are compared to IC₅₀s (nM) derived from BRAMA assay).

A similar comparison for a set of compounds known to have B-Raf inhibitory activity was made between BRAMA and filter-binding assays to follow radiolabeled phosphate incorporation into a kinase-dead derivative of MEK (FIG. 6C). This larger set of compounds also demonstrates a good correlation with BRAMA down to an IC₅₀ of approximately 10 nM. Above that potency, IC₅₀s in the filter binding assay plateau while the BRAMA assay is sensitive to compounds as potent as 0.52 nM. Given the high concentration of B-Raf necessary for the filter binding assay (40 nM) compared to the B-Raf concentration in BRAMA (0.25 nM), these results likely demark the tight-binding limit of the filter binding assay format.

The present studies demonstrate the advantages of the BRAMA assay format over existing B-Raf catalytic assays. BRAMA assays can be run with a B-Raf concentration of 0.25 nM, but this could be reduced further to accommodate the measurement of more potent inhibitors. The BRAMA assay provides information on structure-activity relationships, useful in B-Raf targeted drug discovery efforts.

### REFERENCES

Alessandrini A, Crews C M, Erikson R L. Phorbol ester stimulates a protein-tyrosine/threonine kinase that phosphorylates and activates the Erk-1 gene product. Proc Natl Acad Sci USA. 1992 Sep. 1; 89 (17):8200-4.
Alessi D R, Cohen P, Ashworth A, Cowley S, Leevers S J, Marshall C J. Assay and expression of mitogen-activated protein kinase, MAP kinase kinase, and Raf. Methods Enzymol. 1995; 255:279-90.
Alessi D R, Saito Y, Campbell D G, Cohen P, Sithanandam G, Rapp U, Ashworth A, Marshall C J, Cowley S. Identification of the sites in MAP kinase kinase-1 phosphorylated by p74raf-1. EMBO J. 1994 Apr. 1; 13 (7):1610-9.
Antonsson B, Marshall C J, Montessuit S, Arkinstall S. An in vitro 96-well plate assay of the mitogen-activated protein kinase cascade. Anal Biochem. 1999 Feb. 15; 267 (2):294-9.
Bollag G, Freeman S, Lyons J F, Post L E. Raf pathway inhibitors in oncology. Curr Opin Investig Drugs. 2003 December; 4 (12):1436-41.
Bondzi C, Grant S, Krystal G W. A novel assay for the measurement of Raf-1 kinase activity. Oncogene. 2000 Oct. 12; 19 (43):5030-3.
Burack W R, Sturgill T W. The activating dual phosphorylation of MAPK by MEK is nonprocessive. Biochemistry. 1997 May 20; 36 (20):5929-33.
Crews C M, Erikson R L. Purification of a murine protein-tyrosine/threonine kinase that phosphorylates and activates the Erk-1 gene product: relationship to the fission yeast byr1 gene product. Proc Natl Acad Sci USA. 1992 Sep. 1; 89 (17):8205-9.
Davies H, Bignell G R, Cox C, Stephens P, Edkins S, Clegg S, Teague J, Woffendin H, Garnett M J, Bottomley W, Davis N, Dicks E, Ewing R, Floyd Y, Gray K, Hall S, Hawes R, Hughes J, Kosmidou V, Menzies A, Mould C, Parker A, Stevens C, Watt S, Hooper S, Wilson R, Jayatilake H, Gusterson B A, Cooper C, Shipley J, Hargrave D, Pritchard-Jones K, Maitland N, Chenevix-Trench G, Riggins G J, Bigner D D, Palmieri G, Cossu A, Flanagan A, Nicholson A, Ho J W, Leung S Y, Yuen S T, Weber B L, Seigler H F, Darrow T L, Paterson H, Marais R, Marshall C J, Wooster R, Stratton M R, Futreal P A. Mutations of the BRAF gene in human cancer. Nature. 2002 Jun. 27; 417 (6892):949-54.
Favata M F, Horiuchi K Y, Manos E J, Daulerio A J, Stradley D A, Feeser W S, Van Dyk D E, Pitts W J, Earl R A, Hobbs F, Copeland R A, Magolda R L, Scherle P A, Trzaskos J M. Identification of a novel inhibitor of mitogen-activated protein kinase kinase. J Biol Chem. 1998 Jul. 17; 273 (29):18623-32.
Force T, Bonventre J V, Heidecker G, Rapp U, Avruch J, Kyriakis J M. Enzymatic characteristics of the c-Raf-1 protein kinase. Proc Natl Acad Sci USA. 1994 Feb. 15; 91 (4): 1270-4.
Huang W, Alessandrini A, Crews C M, Erikson R L. Raf-1 forms a stable complex with Mek1 and activates Mek1 by serine phosphorylation. Proc Natl Acad Sci USA. 1993 Dec. 1; 90 (23):10947-51.
King A J, Patrick D R, Batorsky R S, Ho M L, Do H T, Rusnak D W, Takle A K, Wilson D M, Want L, Karreth F, Schaber M D, Voycik J J, Luo L, Lakdawala A S, Adams J L, Smalley K S M, Herlyn M, Tuveson D A, Huang P S. B-Raf(V600E) expression status predicts the anti-tumor response to SB-590885, a potent and selective B-Raf inhibitor. Manuscript in preparation.
Lange-Carter C A, Johnson G L. Assay of MEK kinases. Methods Enzymol. 1995; 255:290-301.
Lee J T Jr, McCubrey J A. Targeting the Raf kinase cascade in cancer therapy—novel molecular targets and therapeutic strategies. Expert Opin Ther Targets. 2002 December; 6 (6):659-78.
Mallon R, Feldberg L R, Kim S C, Collins K, Wojciechowicz D, Hollander I, Kovacs E D, Kohler C. An enzyme-linked immunosorbent assay for the Raf/MEK1/MAPK signaling cascade. Anal Biochem. 2001 Jul. 1; 294 (1):48-54.
McDonald O B, Chen W J, Ellis B, Hoffmnan C, Overton L, Rink M, Smith A, Marshall C J, Wood E R. A scintillation proximity assay for the Raf/MEK/ERK kinase cascade: high-throughput screening and identification of selective enzyme inhibitors. Anal Biochem. 1999 Mar. 15; 268 (2):318-29.
Moll G W Jr, Kaiser E T. Phosphorylation of histone catalyzed by a bovine brain protein kinase. J Biol Chem. 1976 Jul. 10; 251 (13):3993-4000.
Mulcahy P, O'Flaherty M, Jennings L, Griffin T. Application of kinetic-based biospecific affinity chromatographic systems to ATP-dependent enzymes: studies with yeast hexokinase. Anal Biochem. 2002 Oct. 15; 309 (2):279-92.
O'Brian and Ward. Characterization of a Ca2(+)- and phospholipid-dependent ATPase reaction catalyzed by rat brain protein kinase C. Biochemistry. 1990 May 8; 29 (18):4278-82.
Paudel H K, Carlson G M. The ATPase activity of phosphorylase kinase is regulated in parallel with its protein kinase activity. J Biol Chem. 1991 Sep. 5; 266 (25):16524-9.
Prowse C N, Lew J. Mechanism of activation of ERK2 by dual phosphorylation. J Biol Chem. 2001 Jan. 5; 276 (1):99-103.
Soga S, Kozawa T, Narumi H, Akinaga S, Irie K, Matsumoto K, Sharma S V, Nakano H, Mizukami T, Hara M. Radicicol leads to selective depletion of Raf kinase and disrupts K-Ras-activated aberrant signaling pathway. J Biol Chem. 1998 Jan. 9; 273 (2):822-8.
Stokoe D, McCormick F. Activation of c-Raf-1 by Ras and Src through different mechanisms: activation in vivo and in vitro. EMBO J. 1997 May 1; 16 (9):2384-96.
Ward N E, O'Brian C A. The intrinsic ATPase activity of protein kinase C is catalyzed at the active site of the enzyme. Biochemistry. 1992 Jun. 30; 31 (25):5905-11.
Webb M R. A continuous spectrophotometric assay for inorganic phosphate and for measuring phosphate release kinetics in biological systems. Proc Natl Acad Sci USA. 1992 Jun. 1; 89 (11):4884-7.
Wellbrock C, Karasarides M, Marais R. The RAF proteins take centre stage. Nat Rev Mol Cell Biol. 2004 November; 5 (11):875-85.
Xu B, Stippec S, Robinson F L, Cobb M H. Hydrophobic as well as charged residues in both MEK1 and ERK2 are important for their proper docking. J Biol Chem. 2001 Jul. 13; 276 (28):26509-15.
Yan M, Templeton D J. Identification of 2 serine residues of MEK-1 that are differentially phosphorylated during activation by raf and MEK kinase. J Biol Chem. 1994 Jul. 22; 269 (29):19067-73.

We claim:

1. A method of screening a test compound to detect B-Raf inhibitory activity, comprising:
(a) providing a reaction mixture containing human B-Raf, unphosphorylated human MEK-1, and ATP in the presence of a test compound, under conditions that would allow phosphorylation of said MEK-1 by B-Raf in the absence of a B-Raf inhibitor;
(b) monitoring NADH concentration in said reaction mixture over time;
where increased NADH concentrations compared to that which would occur in the absence of any B-Raf inhibitor indicates said test compound has B-Raf inhibitory activity.

2. The method of claim 1 where NADH concentration is detected using Optical Density measurements.

3. The method of claim 1 where said human B-Raf contains a glutamic acid substitution for the valine at position 600 (V600E).
